(19) United States
(12) Patent Application Publication
(10) Pub. No.: US 2012/0040361 A1
(43) Pub. Date: Feb. 16, 2012

(54) METHODS AND COMPOSITIONS FOR DETECTION OF LETHAL SYSTEM AND USES THEREOF

(75) Inventor: Shiaw-Der YANG, Hsinchu (TW)

(73) Assignee: NATIONAL TSING HUA UNIVERSITY, Hsinchu (TW)

(21) Appl. No.: 13/186,100

(22) Filed: Jul. 19, 2011

Related U.S. Application Data
(60) Provisional application No. 61/366,679, filed on Jul. 22, 2010.

(51) Int. Cl. C12Q 1/48; G01N 33/573; G01N 33/574; C12Q 1/68

(52) U.S. Cl. 435/6.12; 435/15; 435/6.18; 435/7.4

(57) ABSTRACT

Provided herein is a method for detecting the presence of lethal system in a patient using the expression of nuclear factor A (NFA) in marker cell. In another aspect, provided herein is a method for predicting if a patient has metastatic potential and is at risk of developing metastasis and for determining a prognosis for the patient.

### CROSS-REFERENCE TO RELATED APPLICATIONS

This Non-provisional application claims the benefit of priority to U.S. Provisional Application No. 61/366,679 filed Jul. 22, 2010, the entire contents of which are hereby incorporated by reference.

### FIELD OF THE INVENTION

The present invention relates to methods and kits useful in identifying and detecting micrometastasis useful for predicting prognosis in various types of cancer patients regardless of the etiological origin of the cancer and uses thereof. The present invention further provides methods and kits useful in predicting if a patient has metastatic potential disease and at risk of developing systemic metastasis.

### BACKGROUND OF THE INVENTION

Most deaths from cancer are due to metastatic disease. However, after producing nearly two million papers on cancer during the last 50 years, we are yet to understand when and how cancer cells metastasize. It is now clear that metastasis is the end product of an evolutionary process between cancer cells and their microenvironment and systemic cancer progression can be due to parallel progression of primary tumors and metastases arising from early disseminated epithelia and mesenchymal-like tumor cell (MTC)/mesenchymal-like tumor stem cell (MTSC) generated by stable reprogramming of epithelial-mesenchymal transition (EMT) via complex inducers provided by both tumor cells and stromal cells. However, the key signaling molecule serving as the main orchestrator to mediate this complex network remains to be established. It is generally accepted that cell culture and animal experiments may not provide accurate models in systemic clinical oncology studies. Although valuable results can be obtained from tumor transplantation experiments, caution needs to be exercised in the simplified interpretation of the overall results and the conclusions need to be validated in models of spontaneously-occurring tumors in cancer patients. For instance, some animal models such as xenotransplantation of cell clines that are derived from advances tumor or metastases and the fidelity of such models to human cancers particularly associated with paracrine, autocrine and endocrine complex network is questionable. This may explain why after producing nearly two million cancer papers, we are yet to understand when and how metastases occur and to learn the underlying mechanisms with sizable impact on cancer mortality. This also explains why the current treatment modalities often failed to cure the cancer patients during the last 50-year comprehensive cancer research. The critical results obtained from laboratory cell cultures and animal experiments definitely should be validated in human clinical samples and in cancer patients' tumors.

The field of molecular, cell and systemic clinical oncology combined has expanded our understanding of cancer as more than a single cancer cell disease. Rather, cancer involves reciprocal interaction and coevolution among conventional cancer cells, host stroma and bone marrow cells (BMC) with chemokines and cytokines network serving as the mediators through pleiotropic signaling mechanisms. Simultaneous targeting not only cancer cell component but also cancer-associated stroma and BMC involved in systemic seeding of premetastatic niche formation, aberrant bone marrow (BM) niche formation and primary endocrine instigation should improve the treatment, overall survival and most importantly the life quality of most cancer patients. However, the most potential molecular signaling target in cell and systemic clinical oncology regarding the vicious cycles of tumor-EMT-stroma coevolution signaling during cancer development and progression remains to be established.

Nuclear factor A (NFA) was originally identified as a specific membrane and cytoplasmic activating factor A of ATP•Mg-dependent protein phosphatase but has subsequently been characterized as a multisubstrate/multifunctional proline-directed protein kinase (PDPK). Due to high sequence homology in kinase domain, NFA was regarded as a subtype of GSK 3 (glycogen synthase kinase 3), and renamed as GSK-3α. Although GSK 3/GSK-3β and NFA/GSK-3α have long been regarded as two closely-related signaling molecule, albeit structurally-similar in kinase domain, they are not functionally-equivalent or -redundant as previously conceived in drosophila and rodents when based on human clinical studies as demonstrated in this application. Moreover, intensive study and most attention have focused on GSK-3β but entitled GSK3 without further specifications in many areas of research and suppression of this kinase may cause tumorigenesis which raises a serious issue concerning how to treat diabetes without causing cancer. As a result, the unique role of NFA has been overlooked for more than a decade. To avoid unnecessary confusions, the term NFA will be used to fully illustrate the pivotal essential role of this novel multisubstrate/multifunctional kinase for comprehensive cancer control.

It has been appreciated that a predictive relationship can exist between the presence of one single unique molecule expression in a cell and the disease status of a patient regardless of the etiology of cancer. Developing universally applicable molecule expression in a cell for determining a patient's risk of developing a beyond curable disease has never been accomplished.

### SUMMARY OF THE INVENTION

The field of molecular, cell and systemic clinical oncology combined has expanded our understanding of cancer as more than a single cancer cell disease. Rather, cancer involves reciprocal interaction and coevolution among conventional cancer cells, host stroma and bone marrow cells (BMC) with chemokines and cytokines network serving as the mediators through pleiotropic signaling mechanisms. Simultaneous targeting not only cancer cell component but also cancer-associated stroma and BMC involved in systemic seeding of premetastatic niche formation, aberrant bone marrow (BM) niche formation and primary endocrine instigation should improve the treatment, overall survival and most importantly the life quality of most cancer patients. In contrast to the previous work on PDPK F_{A}/GSK-3α which was mainly associated with the mainstream cancer research with major focus on conventional cancer cells as described above, the present invention was to examine the role of this signal transducing PDPK in reciprocal interaction and coevolution among conventional cancer cells, host stroma and BMC with chemokines and cytokines network. By using such novel approaches, the aberrant expressions of PDPK F_{A}/GSK-3α in host stroma and BMC were demonstrated to play a determinant and instructional role in determining metastatic potential. Cancer patients if associated with aberrant expressions of PDPK F_{A}/GSK-3α reciprocal interaction and coevolution among conventional cancer cells, host stroma and BMC tend to develop metastatic disease. Thus, the reciprocal interaction and coevolution among conventional cancer cells, host stroma and BMC if associated with aberrant expressions of PDPK F_{A}/GSK-3α was collectively termed “lethal system” in this invention. The lethal system provided herein represents a universally, applicable predictor useful for detection of metastasis useful for monitoring disease status and therapy responses in various types of cancer patients regardless of the etiological origin of the cancer and uses thereof. More particularly, the lethal system is a reliable predictor for determining if a patient has metastasis and at risk of developing metastasis regardless of its etiological origin.

Thus, in one aspect, provided herein is a method for detecting the presence of a cellular expression profile preferably a marker cell in a patient indicative of lethal system, which method comprises obtaining a biological sample from said patient; determining expression of NFA in marker cell in the biological sample wherein the expression of NFA in marker cell of said patient indicates the presence of lethal system, wherein said marker cells is mesenchymal tumor cell (MTC). In some embodiments, the expression of NFA is determined by assaying NFA protein levels such as an immunoassay using antibodies specific for NFA. In other aspects, the expression can be determined by assessing activity, protein, mRNA or DNA level. The biological sample can be bone marrow, cord blood, peripheral blood, tissue sample, ascites, pleural effusions or body fluids.

In one aspect, identification of lethal system in a biological sample is useful for predicting prognosis of cancer patients and predicting if a patient has metastatic potential and at risk of developing metastasis.

In another aspect, provided herein is a diagnostic kit for determining the presence of lethal system in a biological sample comprising at least one reagent for determining expression of NFA in said sample, and printed instructions for assessing the presence of lethal system, packaged together in a container. Further detection reagents may also be included.

Furthermore, in another aspect, provided herein is a method for predicting prognosis of cancer patients and predicting if a patient has metastatic potential and at risk of developing metastasis. The method comprises obtaining a biological sample from said patient; determining expression of NFA in said sample wherein the expression of NFA is used to predict whether the patient is at risk of developing metastasis. In some embodiments, the expression of NFA is determined by assaying NFA protein levels such as an immunoassay using antibodies specific for NFA. In other aspects, the expression can be determined by assessing activity, protein, mRNA or DNA level. The biological sample can be bone marrow, cord blood, peripheral blood, tissue sample, ascites, pleural effusions or body fluids.

If necessary, the cell can be isolated for detection of lethal system highly expressing NFA by specific magnetic beads or flow cell sorter essentially.

These and other objects and features of the invention will become more fully apparent when the following detailed description is read in conjunction with the accompanying figures and examples.

### BRIEF DESCRIPTION OF THE DRAWINGS

FIG. 1 illustrates the NFA lethal system mediates the comprehensive autocrine-paracrine-endocrine signaling interplay network during breast tumor progression. By using coevolution status and NFA expression as an excellent model, we found that the poor outcome breast tumor is accompanied by the release of single individual NFA⁺/vimentin⁺/S100 calcium-binding protein A4(S100A4)/fibroblast-specific protein-1⁺ (FSP-1⁺) large round-shaped migratory mesenchymal-like tumor cell (MTC) at the invasion front (A) and particularly gathering within distant tumor stroma (B) and perivasular area (C) and also within intravasation area (D), providing evidence for a crucial role of NFA in EMT induction and breast tumor progression. Concomitantly, a population of hierarchical NFA⁺ BMCs comprising a rare subset of CD90⁺ mesenchymal/hematopoietic stem/progenitor cells (MSC/HSPC), (E and F) and CD34⁺ HSPC (G) together with a relatively large subset of CD68⁺ macrophages (TAM)(H) and smaller vimentin⁺/FSP-1⁺ spindle-shaped fibroblasts (CAF) (B and C) could be simultaneously detected to coevolve with NFA⁺ MTC as shown in B and C within poor outcome breast tumor stroma. Taken together, the NFA lethal system plays a comprehensive multifunctional role in autocrine-paracrine-endocrine signaling interplay network mediated by EMT-TSCC-BMC through crosstalk among MTC, CAF, TAM, MSC and HSPC during breast tumor progression. NFA immunostaining was developed with DAB, resulting in a red-to-brown color. The BCIP/NBT solution was used to localize large round-shaped vimentin⁺/S100A4/FSP-1⁺ MTC, smaller spindle-shaped CAF, CD90⁺ MSC/HSPC, CD34⁺ HSPC and CD68⁺ TAM, resulting in a blue color. Costaining resulted in a purple-to-black color. Each section was counterstained with methyl green solution, resulting in a green color (original magnification×400).

FIG. 2 depicts the NFA lethal system mediates the comprehensive autocrine-paracrine-endocrine signaling interplay network during lung tumor progression. By using coevolution status and NFA expression as an excellent model, we found that the poor outcome lung tumor is accompanied by the release of single individual NFA⁺/vimentin⁺/S100A4/FSP-1⁺ large round-shaped migratory mesenchymal-like tumor cell (MTC) at the invasion front (A) and particularly gathering within distant tumor stroma (B) and perivascular area (C) and also within intravasation area (D), providing evidence for a crucial role of NFA in EMT induction and lung tumor progression. Concomitantly, a population of hierarchical NFA⁺ BMCs comprising a rare subset of CD90⁺ mesenchymal/hematopoietic stem/progenitor cells (MSC/HSPC), (E and F) and CD34⁺ HSPC (G) together with a relatively large subset of CD68⁺ macrophages (TAM)(H) and smaller vimentin⁺/FSP-1⁺ spindle-shaped fibroblasts (CAF) (B and C) could be simultaneously detected to coevolve with NFA⁺ MTC as shown in B and C within poor outcome lung tumor stroma. Taken together, the NFA lethal system plays a comprehensive multifunctional role in autocrine-paracrine-endocrine signaling interplay network mediated by EMT-TSCC-BMC through crosstalk among MTC, CAF, TAM, MSC and HSPC during lung tumor progression. NFA immunostaining was developed with DAB, resulting in a red-to-brown color. The BCIP/NBT solution was used to localize large round-shaped vimentin⁺/S100A4/FSP-1⁺ MTC, smaller spindle-shaped CAF, CD90⁺ MSC/HSPC, CD34⁺ HSPC and CD68⁺ TAM, resulting in a blue color. Costaining resulted in a purple-to-black color. Each section was counterstained with methyl green solution, resulting in a green color (original magnification×400).

FIG. 3 shows the NFA lethal system mediates the comprehensive autocrine-paracrine-endocrine signaling interplay network during stomach tumor progression. By using coevolution status and NFA expression as an excellent model, we found that the poor outcome stomach tumor is accompanied by the release of single individual NFA⁺/vimentin⁺/S100A4/FSP-1⁺ large round-shaped migratory mesenchymal-like tumor cell (MTC) at the invasion front (A) and particularly gathering within distant tumor stroma (B) and perivasular area (C) and also within intravasation area (D), providing evidence for a crucial role of NFA in EMT induction and stomach tumor progression. Concomitantly, a population of hierarchical NFA⁺ BMCs comprising a rare subset of CD90⁺ mesenchymal/hematopoietic stem/progenitor cells (MSC/HSPC), (E and F) and CD34⁺ HSPC (G) together with a relatively large subset of CD68⁺ macrophages (TAM)(H) and smaller vimentin⁺/FSP-1⁺ spindle-shaped fibroblasts (CAF) (B and C) could be simultaneously detected to coevolve with NFA⁺ MTC as shown in B and C within poor outcome stomach tumor stroma. Taken together, the NFA lethal system plays a comprehensive multifunctional role in autocrine-paracrine-endocrine signaling interplay network mediated by EMT-TSCC-BMC through crosstalk among MTC, CAF, TAM, MSC and HSPC during stomach tumor progression. NFA immunostaining was developed with DAB, resulting in a red-to-brown color. The BCIP/NBT solution was used to localize large round-shaped vimentin⁺/S100A4/FSP-1⁺ MTC, smaller spindle-shaped CAF, CD90⁺ MSC/HSPC, CD34⁺ HSPC and CD68⁺ TAM, resulting in a blue color. Costaining resulted in a purple-to-black color. Each section was counterstained with methyl green solution, resulting in a green color (original magnification×400).

FIG. 4 illustrates the NFA lethal system mediates the comprehensive autocrine-paracrine-endocrine signaling interplay network during colorectum tumor progression. By using coevolution status and NFA expression as an excellent model, we found that the poor outcome colorectum tumor is accompanied by the release of single individual NFA⁺/vimentin⁺/S100A4/FSP-1⁺ large round-shaped migratory mesenchymal-like tumor cell (MTC) at the invasion front (A) and particularly gathering within distant tumor stroma (B) and perivasular area (C) and also within intravasation area (D), providing evidence for a crucial role of NFA in EMT induction and colorectum tumor progression. Concomitantly, a population of hierarchical NFA⁺ BMCs comprising a rare subset of CD90⁺ mesenchymal/hematopoietic stem/progenitor cells (MSC/HSPC), (E and F) and CD34⁺ HSPC (G) together with a relatively large subset of CD68⁺ macrophages (TAM)(H) and smaller vimentin⁺/FSP-1⁺ spindle-shaped fibroblasts (CAF) (B and C) could be simultaneously detected to coevolve with NFA⁺ MTC as shown in B and C within poor outcome colorectum tumor stroma. Taken together, the NFA lethal system plays a comprehensive multifunctional role in autocrine-paracrine-endocrine signaling interplay network mediated by EMT-TSCC-BMC through crosstalk among MTC, CAF, TAM, MSC and HSPC during colorectum tumor progression. NFA immunostaining was developed with DAB, resulting in a red-to-brown color. The BCIP/NBT solution was used to localize large round-shaped vimentin⁺/S100A4/FSP-1⁺ MTC, smaller spindle-shaped CAF, CD90⁺ MSC/HSPC, CD34⁺ HSPC and CD68⁺ TAM, resulting in a blue color. Costaining resulted in a purple-to-black color. Each section was counterstained with methyl green solution, resulting in a green color (original magnification×400).

FIG. 5 illustrates the multifaceted role of the NFA lethal system as a potential target for comprehensive cancer control.

### DETAILED DESCRIPTION OF THE INVENTION

For clarity of disclosure, and not by way of limitation, the detailed description of the invention is divided into the subsections that follow.

### A. Definition

Unless defined otherwise, all technical and scientific terms used herein have the same meaning as is commonly understood by one of ordinary skill in the art to which this invention belongs. All patents, applications, published applications and other publications, and Genebank Accession numbers referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “NFA” refers to the nuclear proline-directed protein kinase FA also known as glycogen synthase kinase-3α. The Genbank Accession numbers for this protein are AAD11986 and AAH27984.

As used herein, “biological sample” refers to any sample from a biologic source, including but not limited to bone marrow, blood, tissue sample, ascites, pleural effusions, body fluids or cell lines.

As used herein, the term “antibody” refers to an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity, e.g., specifically bind NFA.

As used herein, the term “marker cell” refers to cell preferably bone marrow cell (BMC) or cancer-associated fibroblast (CAF) or circulating tumor cell (CTC) or circulating tumor stem cell (CTSC) or epithelial tumor cell (ETC) or epithelial tumor stem cell (ETSC) or hematopoietic stem/progenitor cell (HSPC) or mesenchymal stem cell (MSC) or mesenchymal tumor cell (MTC) or mesenchymal tumor stem cell (MTSC) or tumor-associated macrophage (TAM) which could be recognized by markers. Exemplary markers include, but are not limited to CD34, CD68, CD90, vimentin, fibroblast-specific protein-1 (FSP-1) and S100 calcium-binding protein A4 (S100A4).

As used herein, the term “lethal system” refers to a signaling interplay network preferably epithelial-mesenchymal transition (EMT) or tumor-stroma coevolutional communication (TSCC) associated with aberrant expression of NFA in said marker cell.

As used herein, the term “prognosis” refers to the predicted outcome for patients with a particular disease or condition, such as cancer, after a particular treatment or intervention.

As used herein, the term “metastatic potential” refers to the transmission of cancerous cells from an original site to one or more sites elsewhere in the body.

Unless otherwise indicated, all terms used herein have the same meaning as they would to one skilled in the art and the practice of this invention will be employed, conventional techniques of biochemical and clinical pathological technology, which are within the knowledge of those of skill of the art.

### B. Methods and Kits for Detecting Lethal System

A cell preferably a marker cell associated with aberrant expression of NFA, the lethal system, offers a tool to identify if a patient has metastatic potential and at risk of developing metastasis. In one aspect, identification of a lethal system in a biological sample is useful for monitoring disease status and therapy responses in various types of cancer patients and predicting the development of micrometastasis.

Thus, in one aspect, provided herein is a method for detecting the presence of a cellular expression profile in a patient indicative of lethal system, which method comprises obtaining a biological sample from said patient; determining expression of NFA in cell in said sample wherein the expression of NFA in cell of said patient indicates the presence of lethal system. In some embodiments, the expression of NFA is determined by assaying NFA protein level such as an immunoassay using antibodies specific for NFA. In other aspects, the expression can be determined by assessing activity, protein, RNA or DNA level. The biological sample can be bone marrow, cord blood, peripheral blood, tissue sample, ascites, pleural effusions or body fluids.

In another aspect, provided herein is a kit for determining the presence of lethal system in a biological sample comprising at least one reagent for determining expression of NFA in a cell in said sample, and printed instructions for assessing the relative levels of NFA, packaged together in a container.

Any suitable means of detecting NFA expression may be employed. The expression can be determined by assessing activity, protein, RNA or DNA levels in cell from a biological sample. For example, an immunoassay using an antibody specific for NFA may be employed. Suitable means include, but are not limited to immunohistochemical analysis, immunocytochemical analysis, flow cytometry analysis, Western blot analysis, Northern blot analysis, RT-PCR and phosphorylation assays on specific substrates. With immunohistochemical staining techniques, a cell sample is prepared, typically by dehydration and fixation, followed by reaction with labeled antibodies specific for the gene product coupled, where the labels are usually visually detectable, such as enzymatic labels, florescent labels, luminescent labels, and the like.

According to one embodiment, tissue samples are obtained from patients and the samples are embedded then cut to e.g. 3-5 μm, fixed, mounted and dried according to conventional tissue mounting techniques. The fixing agent may comprise formalin. The embedding agent for mounting the specimen may comprise, e.g., paraffin. The samples may be stored in this condition. Following deparaffinization and rehydration, the samples are contacted with an immunoreagent comprising an antibody specific for NFA. The antibody may comprise a polyclonal or monoclonal antibody. The antibody may comprise an intact antibody, or fragments thereof capable of specifically binding NFA protein. Appropriate polyclonal antisera or other antibody may be prepared by immunizing appropriate host animals with NFA protein, or a suitable fragment thereof, and collecting and purifying the antisera according to conventional techniques known to those skilled in the art. Monoclonal or polyclonal antibodies, preferably monoclonal, specifically reacting with NFA, may be made by methods well known in the art. Also, recombinant antibodies may be produced by methods known in the art, including but not limited to. Monoclonal antibodies with affinities of 108 M−1, preferably 109 to 1010 M−1 or more, are preferred.

The antibody either directly or indirectly bears a suitable detectable label. Alternatively, the detectable label can be attached to a secondary antibody, e.g., goat anti-rabbit IgG, which binds the primary antibody. Frequently, the polypeptides and antibodies are labeled by joining, either covalently or noncovalently, a substance which provides a detectable signal. Suitable labels include, but are not limited to, radionuclides, enzymes, substrates, cofactors, inhibitors, fluorescent agents, chemiluminescent agents, magnetic particles and the like.

Any suitable means can be used to obtain a biological sample from a patient. A biological sample can be peripheral blood, cord blood, bone marrow, tissue sample, ascites, pleural effusions or body fluids.

Kits for determining if a patient has micrometastasis and at risk of developing metastasis will include at least one container sized to house at least one reagent useful in determining expression of NFA in cell as defined herein, and printed instructions for assessing whether or not cells in a biological sample contain one or more lethal system. As used herein, the term “reagent” means any compound, composition or biological agent (i.e., samples, aliquots or “doses” of cells, antibodies, etc.) useful in carrying out any method provided herein, including but not limited to antibodies for NFA, buffers and carriers useful in isolating and preparing cells and/or membranes for analysis and treatment, buffers and carriers useful in carrying out saturation and competition binding assays, and radioactive and non-radioactive labeling compounds. The printed instructions will also include instructions for correlating the results of the tests with the lethal system phenotype.

### C. Embodiments

Unless otherwise indicated in the specific embodiments, all immunohistochemical analysis, immunophenotyping analysis, immunocytochemical analysis and statistical analysis followed the below methods.

Patients: Clinicopathologic data and the specimens used for immunohistochemical analysis were obtained through a detailed retrospective review of the medical records of patients who had undergone initial tumor resection for cancer at National Taiwan University Hospital, Taipei, Taiwan, between 1987 and 2004. Surgically resected specimens were fixed in 10% formalin and routinely processed for paraffin embedding. Serial sections were stained with hematoxylin and eosin for histological evaluation. Patients were observed until April, 2006. The study was approved by the Institution's Surveillance and Ethics Committee.

Production, Identification and Characterization of Specific Anti-NFA Antibody. The peptide QSTDATPTLTNSS (SEQ NO.1), corresponding to the carboxyl terminal region from amino acids 471 to 483 of the sequence of NFA was synthesized by peptide synthesizer (model 9050, Milligen, Bedford, Mass.). The cysteine residue was added to the NH2 terminus in order to facilitate coupling of the peptide to bovine serum albumin according to the procedure described by Reichlin (1980) using glutaraldehyde as the cross-linker. The antibody production has been through affinity purification and the recognition that could be blocked by the C-terminal peptide from amino acids 471-483 of NFA to demonstrate the immunospecificity of this anti-NFA antibody.

Immunohistochemial Analysis. Tissue sections (5 μm) of formalin-fixed, paraffin-embedded tissue containing tumor that showed the maximum extent of tumor cells were dewaxed in xylene and rehydrated in graded concentrations of ethanol. Endogenous peroxidase was blocked with 3% hydrogen peroxide followed by bovine serum albumin blocking for 5 minutes. The slides were next incubated with primary antibody (2 μg/mL) NFA, OPN, IL-6, TGF β, TNF α, tissue factor, VEGF diluted in 0.05 M Tris buffer, pH 7.4, at 4° C. for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). For double staining, slides were incubated in DS-enhancer (Zymed, San Francisco, Calif.) at room temperature for five minutes after quenching the enzyme reaction to prevent the interaction between two staining system. Then, slides were incubated with specific marker as indicated for one hour at room temperature. After washing, slides were incubated with anti-mouse alkaline phosphatase for 30 minutes at room temperature. BCIP/NBT solution was used for visualization of the bound specific marker such as for vimentin/S100A4/FSP-1, CD90, CD34 and CD68. NFA, OPN, IL-6, TGFβ, TNFα, tissue factor, VEGF immunostaining was developed with DAB, resulting in a red-to-brown color. The BCIP/NBT solution was used to localize large round-shaped vimentin+/S100A4/FSP-1+ MTC and smaller spindle-shaped CAF, CD90+ MSC/HSPC, CD34+ HSPC and CD68+ TAM, resulting in a blue color. Costaining resulted in a purple-to-black color. Single staining was counterstained with hematoxylin and double staining was counterstained with methyl green solution.

Immunocytochemical analysis. On average 1×10⁶ cells will be cytocentrifuged onto polylysine-coated slides at 700 rpm for 3 minutes at room temperature (Kubota 5200, Japan). Before staining, the cytospots will be fixed with 3.7% paraformaldehyde for 15 minutes and treated with 0.2% triton X-100 for 90 seconds. Endogenous peroxidase will be blocked with 3% hydrogen peroxide followed by bovine serum albumin blocking for 10 minutes. The slides will be incubated with anti-NFA antibody (2 μg/mL) diluted in 0.05 M Tris buffer, pH 7.4, at 4° C. for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). For double staining, slides were incubated in DS-enhancer (Zymed, San Francisco, Calif.) at room temperature for five minutes after quenching the enzyme reaction to prevent the interaction between two staining system. Then, slides were incubated with specific marker as indicated for one hour at room temperature. After washing, slides were incubated with anti-mouse alkaline phosphatase for 30 minutes at room temperature. BCIP/NBT solution was used for visualisation of the bound specific marker such as for vimentin/S100A4/FSP-1, CD90, CD34 and CD68. NFA, OPN, IL-6, TGFβ, TNFα, tissue factor, VEGF immunostaining was developed with DAB, resulting in a red-to-brown color. The BCIP/NBT solution was used to localize large round-shaped vimentin⁺/S100A4/FSP-1⁺ MTC and smaller spindle-shaped CAF, CD90⁺ MSC/HSPC, CD34⁺ HSPC and CD68⁺ TAM, resulting in a blue color. Costaining resulted in a purple-to-black color. Single staining was counterstained with hematoxylin and double staining was counterstained with methyl green solution.

The following examples are offered to illustrate but not to limit the invention.

### Example 1

The cancer patients if associated with the NFA⁺ tumor-EMT-stroma-BMC lethal system had poor outcome even after aggressive and/or potentially curative treatments.

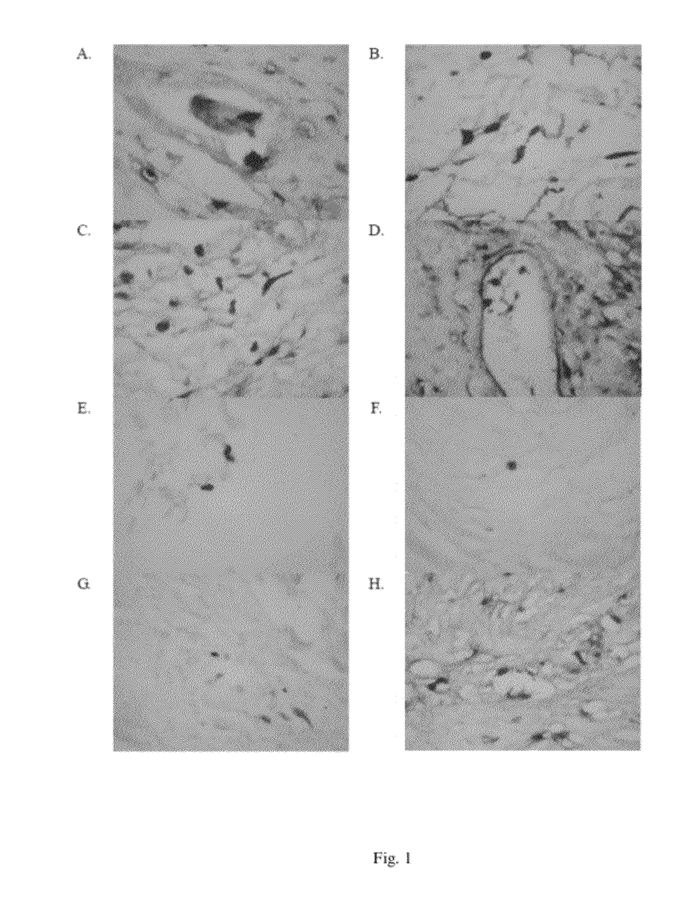

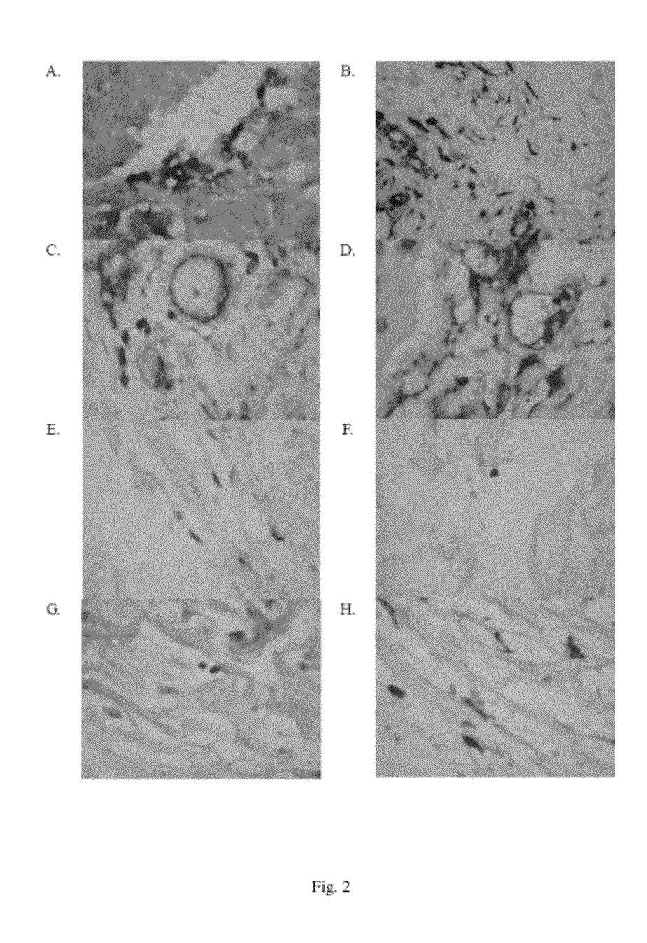

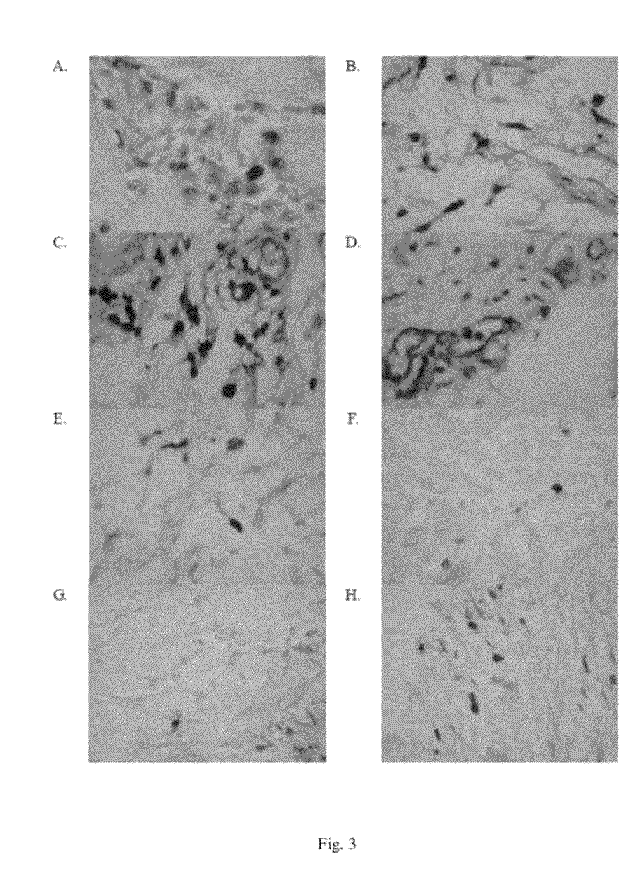

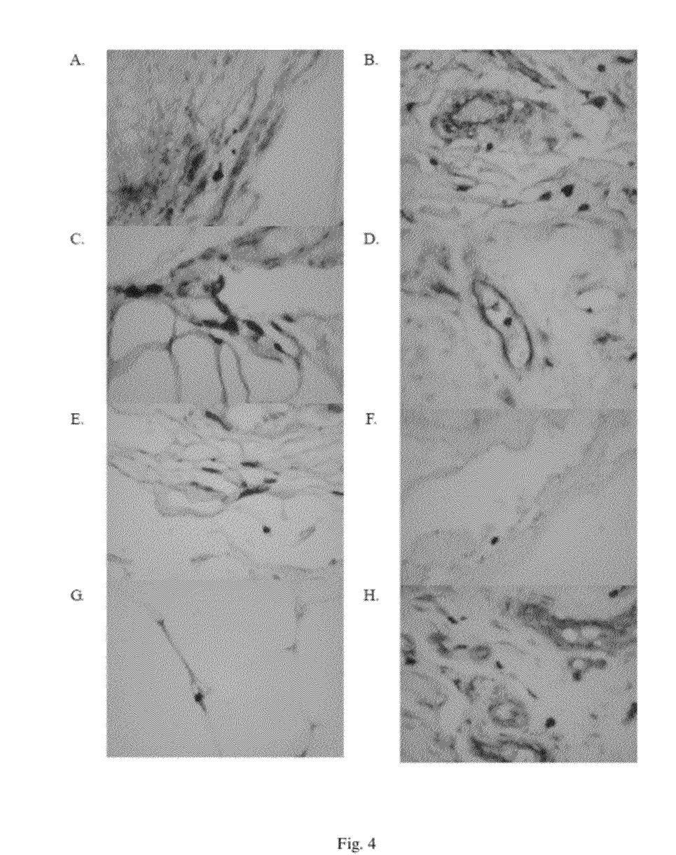

By using coevolution status and NFA expression as an excellent model, we found that the poor outcome breast tumor is accompanied by the release of single individual NFA+/vimentin+/S100A4/FSP-1+ large round-shaped migratory MTC at the invasion front (FIG. 1A) and particularly gathering within distant tumor stroma (FIG. 1B) and perivasular area (FIG. 1C) and also within intravasation area (FIG. 1D), providing evidence for a crucial role of NFA in EMT induction and breast tumor progression. It is now clear that the mesenchymal state through EMT endows cancer cells with migratory and invasive properties, induces cancer stem cell properties, prevents apoptosis and senescence and contributes to immunosuppression and multiresistances to chemotherapy, immunotherapy and targeted therapy. Thus, the mesenchymal state of cancer cells contributes the metastatic and stemness potential of cancer cells for initiation and promotion of cancer progression and metastasis, which is essential and critical for tumor cell detachment, migration, invasion and metastatic dissemination and spread (FIG. 1) Concomitantly, a population of hierarchical NFA+BMCs comprising a rare subset of CD90+ mesenchymal/hematopoietic stem/progenitor cells (MSC/HSPC), (FIGS. 1E and F) and CD34+ HSPC (FIG. 1G) together with a relatively large subset of CD68+ macrophages (TAM)(FIG. 1H) and smaller vimentin+/FSP-1+ spindle-shaped cancer associated fibroblasts (CAF) (FIGS. 1B and C) could be simultaneously detected to coevolve with NFA+MTC as shown in FIGS. 1B and C within poor outcome breast tumor stroma. As further demonstrated in FIGS. 1B and C, coevolution and crosstalk between NFA+vimentin+/S100A4/FSP-1+MTC (large round-shaped) and NFA+vimentin+/S100A4/FSP-1+CAF (smaller spindle-shaped) could be frequently detected within tumor stroma and perivascular area associated with poor clinical outcome. The NFA-mediated EMT induction apparently served as a bridge to initiate the coevolution and crosstalk between NFA+MTC and NFA+CAF to promote cancer progression via the prolonged paracrine interactions between tumor cells and stromal cells as demonstrated in FIG. 1. Taken together, the results provide the comprehensive clinical evidence to support the current paradigm that tumor-stroma coevolution and communication play an important role in tumor progression. NFA within tumor stroma apparently plays a predominant role in determining the poor outcome of breast cancer patients as demonstrated above. Taken together, the results further demonstrate the crucial role of NFA in tumor-stroma coevolution and crosstalk involved in breast tumor progression. By using NFA as a novel probe, we found that EMT, tumor-stroma coevolutional communication (TSCC) and BMC are all involved in determining breast tumor progression and metastasis and poor outcome of the breast cancer patients, which is in agreement with the current paradigm that EMT, TSCC and BMC may play a crucial role in cancer progression. Thus, this invention provides the NFA lethal system for comprehensive cancer control of tumor-EMT-stroma-BMC-mediated vicious cycle which is critical for breast tumor development and progression. Similar observations could also be extended to poor outcome tumors of lung (FIG. 2), stomach (FIG. 3) and colorectum (FIG. 4). The NFA-tumor-EMT-stroma-BMC-mediated vicious cycle turned out to be the most fatal and ubiquitously-expressed system in the cancer patients with poor outcome even after potential curative and/or aggressive treatments. Thus this invention provides a molecular, cell and systemic lethal system for comprehensive cancer control.

### Example 2

### The NFA Lethal System is a Potential Target for Prognosis of a Cancer Patient

In a large cohort study, more than 50% of the poor outcome breast cancer patients (44/74) exhibited the NFA lethal system as described above. On the other hand, the breast cancer patients if associated with the NFA lethal system all failed to have good outcome after the treatment and in a population of 67 good outcome breast cancer patients, no patient exhibited the NFA lethal system. Similarly, ˜56% (44/78) of the poor outcome lung cancer patients exhibited the NFA lethal system and no false positive case could be detected in a population of 53 good outcome lung cancer patients. Similarly, ˜67% (61/91) of the poor outcome GI cancer patients exhibited the NFA lethal system and all had poor outcome after the treatment; no false positive case could be detected in a total of 94 good outcome lung cancer patients after the treatment. Collectively, in a total of 457 cancer patients comprising 214 good outcome and 243 poor outcome cases, more than 60% (149/243) poor outcome patients exhibited the NFA lethal system as described in FIGS. 1-4 and all had poor outcome after the treatment. None of the 214 good outcome tumors exhibited the NFA lethal system. It is noted that the major population of poor outcome patients were associated with bone metastasis. Taken together, the results demonstrate a crucial role of the NFA lethal system predominantly and exclusively in determining the poor clinical outcome of more than 60% of the cancer patients for comprehensive cancer control (Table 1). The poor outcomes of many cancer patients apparently were predominantly determined by both NFA⁺ BMC and particularly NFA⁺ MTC characteristic of metastatic mesenchymal-like cancer stem cells with multiresistances to immune surveillance, apoptosis, premature senescence, chemotherapy, immunotherapy and current targeted therapy. On the other hand, upregulations of various potential EMT inducers within NFA⁺ tumor stroma described above may cause stable reprogramming of EMT-like processes to maintain the NFA⁺ tumor stem cells at a mesenchymal state with metastatic potential. Taken together, all the results represent the molecular, cellular and systemic action mechanisms to explain why one single individual NFA⁺/FSP-1⁺ large round-shaped MTC as shown in FIGS. 1D-4D is sufficient to predominantly determine the poor outcomes of various types of cancer patients. Thus, NFA represents a newly-described, previously-undiscovered signaling target that plays a pivotal role in the stable maintenance of the mesenchymal state of tumor stem cells involved in cancer progression and metastasis.

TABLE 1

| | | | Lethal system |
|---|---|---|---|
| | | Total poor | detection rate of |
| | Poor outcome patients | outcome | poor outcome |
| | with lethal system | patients | patients (%) |
| breast cancer | 44 | 74 | 59.5 |
| lung cancer | 44 | 78 | 56.4 |
| GI cancer | 61 | 91 | 67.0 |
| all cancer | 149 | 243 | 61.3 |

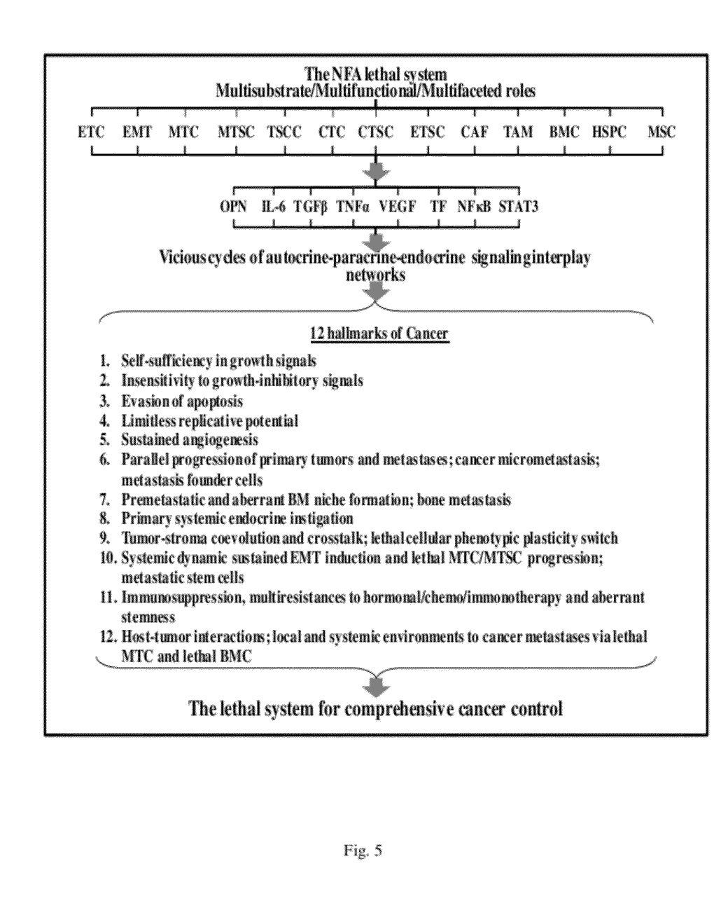

In conclusion, it is now clear that comprehensive cancer control requires simultaneous targeting on autocrine, paracrine and systemic endocrine actions of tumor-EMT-stroma-BMC coevolution signaling. Thus, the present invention provides core technology that could simultaneously target 12 hallmarks of cancer including stable reprogramming of EMT induction, antiapoptosis, premetastatic niche formation, systemic immunosuppression, aberrant tumor-stroma coevolution and crosstalk, cancer-related inflammation, aberrant stemness, aberrant bone marrow niche formation, bone metastasis and primary systemic endocrine instigation for comprehensive cancer control (FIG. 5).

In summary, the tumor cells if associated with NFA predominantly and exclusively exist in a mesenchymal state which is associated with tumor cell migration, invasion, angiogenic switch, immunosuppression, prevention of premature senescence and apoptosis, induction of cancer stemness and multiresistances to chemotherapy, immunotherapy and targeted therapy and poor outcome correlation. Metastasis formation from primary epithelial tumors progresses through various stages including generation of circulating tumor stem cells (CTSC) and derivatives CTCs. Up to now, however, there is no reported unique tumor marker that is specific enough to detect the predominant and determinant rare CTCs to predict metastasis during and after treatment. Moreover, the current CTC markers have been detected in normal blood cells and many false positives have been generated. Thus, immunodetection techniques have mostly examined the presence of cells in the blood expressing epithelial marker and more particularly cytokeratins. For instance, an advanced standardized commercially available system, the Cell Search system developed by Johnson & Johnson USA was an automated device based on EpCAM immunomagnetic purification and cytokeratin staining. This Cell Search technology recently has been approved by FDA for the blood analyses of CTCs in patients with metastatic breast, colorectal and prostate cancers. However, the recent ASCO consensus survey of tumor markers available for potential use technique concluded that the current monitoring for the levels of CTCs was not yet mature for clinic use. The current core technology to establish the predictive/prognostic/diagnostic values of CTCs remains to be improved. As presented above, the cancer patients if associated with NFA⁺ vimentin⁺/FSP-1⁺ large-round-shaped MTC within perivascular area all failed to have good outcome even after potentially curative treatment. In contrast, all good outcome patients exhibited no such type of MTC. It is now clear that MTC is a potential provider of CTC and CTSC. Thus, this invention provides method and composition to detect the most potential MTC, MTSC, CTC and CTSC to predominantly predict micrometastasis and poor outcome of the cancer patients even after potentially curative treatment. In comparisons with the bipolar actions of most EMT inducers such as Twist, Snail, Slug, TGF β 1, TNF α, VEGF and IL-6 which were involved in both normal and pathological processes, the NFA lethal system represents a novel target to revert EMT without damaging normal physiological functions. Thus, this invention provides method and composition of the lethal system for comprehensive cancer control including prediction, prevention, personalized healthcare and palliation prior to and during cancer development and progression and particularly micrometastasis and bone metastasis (FIG. 5).

In terms of the above, the present invention provides a method of predicting if a patient has micrometastasis and at risk of developing metastasis.

What is claimed is:

1. A method for detecting presence of a lethal system in a patient comprising:
obtaining a biological sample from the patient;
determining a marker cell in said biological sample; and
determining expression of NFA in said marker cell;
wherein the expression of NFA in said marker cell in the biological sample indicates the patient has the lethal system.

2. The method of claim 1, wherein said marker cells is selected from the group consisting of: bone marrow cell (BMC), cancer-associated fibroblast (CAF), circulating tumor cell (CTC), circulating tumor stem cell (CTSC), epithelial tumor cell (ETC), epithelial tumor stem cell (ETSC), cancer stem cell (CSC), tumor propagating cell (TPC), hematopoietic stem/progenitor cell (HSPC), mesenchymal stem cell (MSC), mesenchymal tumor cell (MTC), mesenchymal tumor stem cell (MTSC), tumor-associated macrophage (TAM) and myeloid cell.

3. The method of claim 1, wherein said marker cells is mesenchymal tumor cell (MTC).

4. The method of claim 1, wherein said biological sample is selected from the group consisting of: bone marrow, cord blood, peripheral blood, tissue sample, ascites, pleural effusions and body fluids.

5. The method of claim 1, wherein said expression of NFA is determined by assessing NFA protein, mRNA, DNA or activity level.

6. A method of predicting prognosis of a cancer patient, comprising:
obtaining a biological sample from the cancer patient;
determining a marker cells in said biological sample; and
determining expression of NFA in said marker cell;
determining the patient has a poor prognosis by identifying the expression of NFA in said marker cell in the biological sample.

7. The method of claim 6, wherein said marker cells is selected from the group consisting of bone marrow cell (BMC), cancer-associated fibroblast (CAF), circulating tumor cell (CTC), circulating tumor stem cell (CTSC), epithelial tumor cell (ETC), epithelial tumor stem cell (ETSC), cancer stem cell (CSC), tumor propagating cell (TPC), hematopoietic stem/progenitor cell (HSPC), mesenchymal stem cell (MSC), mesenchymal tumor cell (MTC), mesenchymal tumor stem cell (MTSC), tumor-associated macrophage (TAM) and myeloid cell.

8. The method of claim 6, wherein said biological sample is selected from the group consisting of: bone marrow, cord blood, peripheral blood, tissue sample, ascites, pleural effusions and body fluids.

9. The method of claim 6, wherein said expression of NFA is determined by assessing NFA protein, mRNA, DNA or activity level.

10. A method of predicting metastatic potential of a cancer patient, comprising:
obtaining a biological sample from said cancer patient;
determining a marker cell in said biological sample; and
determining expression of NFA in said marker cell;
wherein the expression of NFA in said marker cell in the biological sample indicates a metastatic potential of the cancer patient.

11. The method of claim 10, wherein said marker cells is selected from the group consisting of bone marrow cell (BMC), cancer-associated fibroblast (CAF), circulating tumor cell (CTC), circulating tumor stem cell (CTSC), epithelial tumor cell (ETC), epithelial tumor stem cell (ETSC), cancer stem cell (CSC), tumor propagating cell (TPC), hematopoietic stem/progenitor cell (HSPC), mesenchymal stem cell (MSC), mesenchymal tumor cell (MTC), mesenchymal tumor stem cell (MTSC), tumor-associated macrophage (TAM) and myeloid cell.

12. The method of claim 10, wherein said biological sample is bone marrow, cord blood, peripheral blood, tissue sample, ascites, pleural effusions or body fluids.

13. The method of claim 10, wherein said expression of NFA is determined by assessing NFA protein, mRNA, DNA or activity level.


SEQUENCE LISTING (1 sequence)

SEQ ID NO: 1 - protein, 13 aa, Artificial Sequence
  Specific Anti-NFA Antibody
QSTDATPTLTNSS